(19) United States
(12) Patent Application Publication
(10) Pub. No.: US 2014/0229192 A1
(43) Pub. Date: Aug. 14, 2014

(54) SYSTEM FOR TRACKING TIME-LAPSED HEALTH CHANGES

(71) Applicants: Justin Zastrow, Scottsdale, AZ (US); William Birgen, Tempe, AZ (US)

(72) Inventors: Justin Zastrow, Scottsdale, AZ (US); William Birgen, Tempe, AZ (US)

(21) Appl. No.: 14/178,769

(22) Filed: Feb. 12, 2014

Related U.S. Application Data
(60) Provisional application No. 61/763,601, filed on Feb. 12, 2013.

(51) Int. Cl. A61B 5/103; G06F 19/00; A61B 5/00

(52) U.S. Cl. CPC A61B 5/1032; A61B 5/0015; A61B 5/441; A61B 5/448; A61B 5/45; G06F 19/3418
USPC 705/2; 600/300; 600/306

(57) ABSTRACT

Generally, the present invention is incorporated in a system of interconnected software, devices (e.g., hardware, viewing screens), and databases (the “system”) that receive, log and score a series of objective data over time in order to generate an numerical score (or index). The data can be almost anything, a color, a dimension, the angle between two objects, or other objective relationships that can change over time. For health issues, the data could be a skin mole, teeth color, hair color or recession, various diseases and so forth. This invention is useful for a user who wants to track one or more health issues. In addition, this invention is useful for a seller of goods and services related to the tracked health issue.

### CROSS-REFERENCE TO RELATED APPLICATIONS

The present application claims priority to co-pending U.S. provisional patent application entitled “System For Tracking Time-Lapsed Health Changes” having Ser. No. 61/763,601, filed on Feb. 12, 2013, which is entirely incorporated herein by reference.

### FIELD OF THE INVENTION

The present invention relates to a system for tracking health changes, and more specifically to tracking heath changes over a period of time.

### DESCRIPTION OF RELEVANT ART

Many health issues evolve slowly. So slowly, that sometimes the changes are imperceptible. Other times, the changes would be perceptible but the previous stages are forgotten or simply blend in with the present. For example, a skin mole can change from a benign skin mole to a problematic mole over time, but the changes can be difficult to perceive until it is too late. Likewise, changes in skeletal posture can degrade imperceptibly slowly.

In addition, many cosmetic health issues change over time, too. Teeth stains and gum recession, for example, can develop over years and decades. Hair color, thinning and recession can change over time. Skin color changes take time. Broken bones, chicken pox, diseases or anything in the medical field may also change over time. It may be desirable for a person to track time-lapsed health changes. It also may be desirable for business to be able to market goods and services directly to those who are tracking a given issue.

Currently, no system is known for objectively tracking and analyzing time-lapsed health changes. In this light, what is needed is a method for tracking the progress of health issues and making the changes easy to comprehend. A method for business to directly market goods and services to those who keep track of relevant health issues is also desirable.

### SUMMARY OF THE INVENTION

The structure, overall operation and technical characteristics of the present invention will become apparent with the detailed description of preferred embodiments and the illustration of the related drawings as follows.

Generally, the present invention is incorporated in a system of interconnected software, devices (e.g., hardware, viewing screens), and databases (the “system”) that receives, logs and scores a series of objective data over time in order to generate an numerical score (or index). The data can be almost anything, a color, a dimension, the angle between two objects, or other objective relationships that can change over time. For health issues, the data could be a skin mole, teeth color, hair color or recession, various diseases and so forth.

A numerical score may be easy to comprehend, but other formats may also be adopted, such as a percentage or a number of dots. In addition, how often the user wants to keep the records may be the user's choice or an embodiment may provide a reminder mechanism. The score and the timestamp are recorded in a database for future use. Other relevant information may also be recorded, such as a picture or a brief note. Thus, the user may see how the score gradually changes and learn how the health issue evolves.

Other elements of the invention may be incorporated in optional sell-side modules that inform sell-side subscribers that a user may be interested in purchasing relevant goods and services. Sell-side subscribers could use this information to direct marketing efforts to targeted audiences including registered software information. One embodiment may allow the user or software company to select the health issue of interest, such as skin cancer, teeth whitening, skin tanning, and so forth. Once the user's interest is known, the system may match the users with relevant sell-side subscribers.

The geographical distance between the user and a sell-side subscriber may also be used as a criterion for matching the user with the sell-side subscribers. The user's location may be determined by a mail address provided by the user or an IP address or the location of the user's mobile device that may be acquired without user input via global positioning (GPS). Another embodiment may also inform users of relevant goods and services by passively receiving or actively retrieving advertisements from sell-side subscribers nearby.

One object of the invention is to track health changes by capturing objective data: either by color or by measuring geometric relationships (e.g. postural/skeletal changes).

Another object of this invention is to create an index of color and save the index for tracking the change in colors recorded at different time.

Another object of this invention is to allow sell-side subscribers to more readily connect with relevant users, including the deployment of social media tools.

### BRIEF DESCRIPTION OF THE DRAWINGS OR PICTURES

FIG. 1 illustrates one embodiment of the system for tracking objective health changes and matching a user and a seller-side subscriber based on user interest and geographic area.

FIG. 2 illustrates a flow chart of an embodiment for recording skin tan progress.

FIG. 3 illustrates a user interface of an embodiment for choosing a tanning environment and setting the time amount.

FIG. 4 illustrates a user interface of an embodiment for taking a picture of the user's tan and showing a score of the tan.

FIG. 5 illustrates a user interface of an embodiment for picking a color/goal/target/objective.

FIG. 6 illustrates a user interface of an embodiment for showing tan sessions taken and tracking the tanning progress.

FIGS. 7a & 7b show how an embodiment connecting goods and service providers with target consumers.

### DESCRIPTION OF THE PREFERRED EMBODIMENTS

One preferred embodiment is a system for tracking health changes by transforming a color into a score and then saving the score for future reference. In addition, the preferred embodiment also incorporates a method of bridging consumer/producer gap by informing local sell-side subscribers that a potential consumer has come into their service regions. The embodiment may also inform a user of local providers of goods and services of interest. Another preferred embodiment comprises a system specifically for tan-loving users to track their tanning progress. The embodiment may also match the tan-loving users with tan salons or tanning product retailers.

In addition, the color-change embodiment described below can be adapted to other objectively measured criteria, such as distance changes or angular changes, such as for tracking postural/skeletal changes.

Color-Change Tracking Embodiment

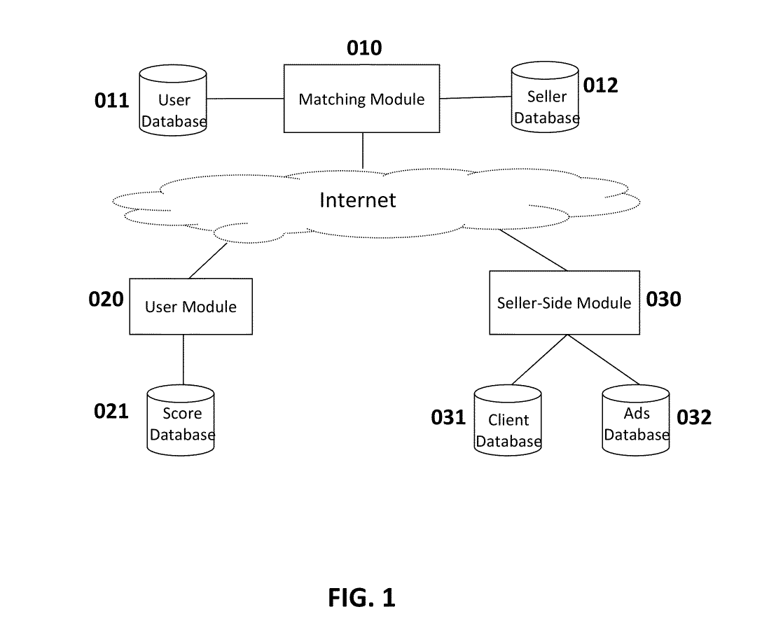

The first embodiment is a system illustrated in FIG. 1. The User Module 020 receives objective data from a user and generates a numerical score based on the input data. The objective data includes the user's interest (or purpose of using this system) and a color. The color may be picked by the user manually or retrieved from a photograph of the user's skin mole, teeth, hair, and so on. The score reflects the relative distance between the color and a predefined goal and/or target color. After the score is determined, the score and a timestamp are recorded in the Score Database 021. If the user wants to review the scores, the User Module 020 retrieves from the database 021 and shows historical data to the user.

The User Module 020 may also communicate with a remote server with a Matching Module 010. User information including the user's interest and location is transmitted to the Matching Module 010. The communication may be periodic or triggered by an event such as a change in the user's location.

The Matching Module 010 in FIG. 1 maintains a Seller Database 012 containing goods and services provided by sell-side subscribers, the sell-side subscribers' contact information, and their corresponding service areas. A sell-side subscriber may be a tan salon, a dermatology clinic, a dentist clinic, a beauty salon, or a retailer of related goods. When user information is received, the Matching Module 010 matches the user with at least one sell-side subscriber that provides the goods or services related to the user's interest and serves the area near the user. A sell-side subscriber may provide goods and services in multiple service categories, therefore not limited to a certain user interest. For example, a tanning product retail store that sells tanning sprays and sun screens may be related to both tanning and skin cancer prevention.

The geographical distance between the user and a sell-side subscriber may also be a criterion for the Matching Module 010. The maximum distance between a user and a matched sell-side subscriber may be decided by the user or predefined by the system—either the User Module 020 or the Matching Module 010. Additional matching criteria may be used. For example, a sell-side subscriber may choose a specific geographic area for offering its goods and services. After matching criteria are determined, the Matching Module 010 provides the user information to a Sell-Side Module 030 for a matched sell-side subscriber. The Matching Module 010 may also maintain its own User Database 011 for saving the user information for other uses.

A Sell-Side Module 030 that receives user information from the Matching Module 010 may inform the sell-side subscriber of the information of its potential customer by any means such as an email or a pop-up message on a computer. The sell-side subscriber may then determine a course of action. Alternatively, the module 030 may automatically add the user information in its Client Database 031. Then the Sell-Side Module 030 retrieves relevant marketing materials from its Ads Database 032 based on the user's interest and forwards the ads to the user. The ads may be sent to the potential customer by mail, email, internet, text message, or any means for communication.

Even though the system in FIG. 1 is described in detail, persons skilled in the relevant system architecture, structure, and data flow would know that this system is only one implementation of the invention, and various modifications exist. The Matching Module 010 may include a software program executed on the same device with the User Module 020 or the Sell-Side Module 030. The Sell-Side Module 030 may not have a Client Database 031 or Ads Database 032 and sends a predefined message to the target consumer without saving the user information. The User Module 020 may also inform its user of relevant goods and services by passively receiving or actively retrieving advertisements from Sell-Side Modules 030 of matched sell-side subscribers nearby.

In sum, various embodiments may be adopted as long as they can generate and save a score for a user to track health issues and optionally allow sell-side subscribers to find target consumers that are tracking health issues related to their goods or services. To further illustrate how the invention may work, below is another embodiment specifically designed for tracking tanning progress.

Tanning Embodiment

Tanning has been in fashion in the western world for over a century. Modern tan lovers darken their skin tones by traditional sun tanning, tanning beds or by using sunless tanning products. When a person actively tans, many factors such as weather condition and the person's natural skin tone affect the time required and the tan that person actually gets. It may take multiple tan sessions to get the tan a person wants. Keeping track of tanning progress may show how each tan session works for a tan lover and help the tan lover to evaluate how many more tan sessions may be needed. Therefore, an effective method for showing the tan degree and the progress is desired.

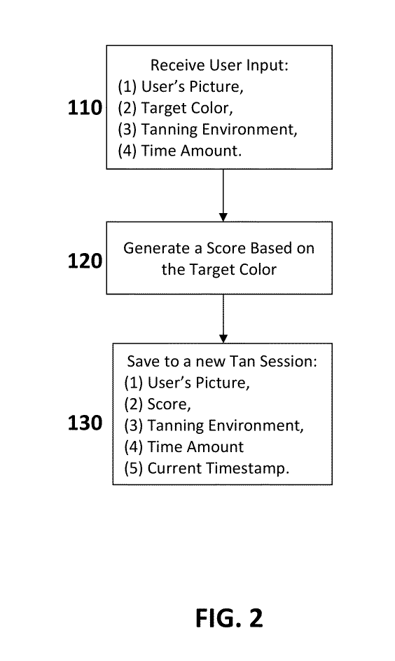

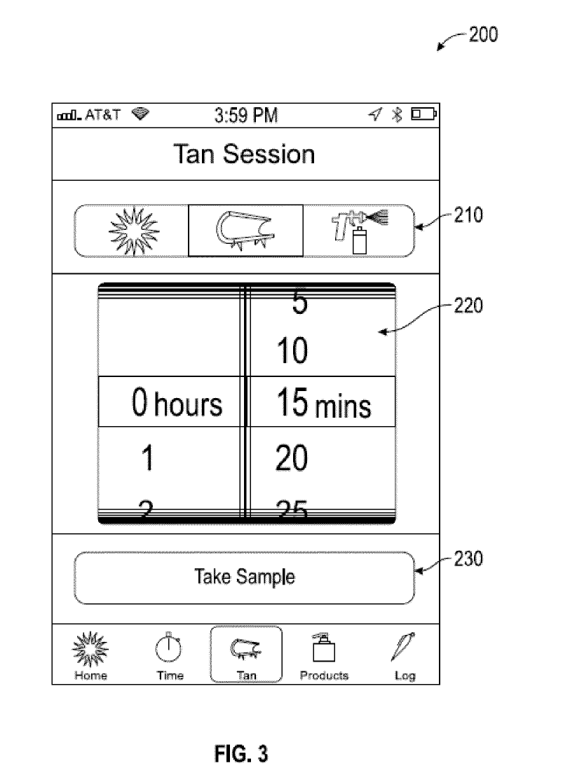

A tanning embodiment comprises a system for receiving user input of skin color and generating a tanning score (i.e., how dark the user's skin is). The system contains a mobile device with user interfaces 200, 300, 400, and 500 (a “user module”) as shown in FIGS. 3-6. The user module allows a user to track the tanning progress, as illustrated in FIG. 2. The system may also contain a server that maintain a list of sell-side subscribers and matches the user with sell-side subscribers (a “matching server”), like the Matching Module 010 in FIG. 1. The system may in addition have another server of a sell-side subscriber that provides goods or services that the tan-loving user may be interested (a “sell-side subscriber server”), like the Sell-Side Module 030 in FIG. 1.

In FIG. 2, the system receives user input 110 where a user takes a picture of his or her tan, and selects a target skin color (a color that is close to the user's tan), the tanning environment, and the time amount that the tan session took. The system then generates a tanning score 120, a numerical value that indicates the relative distance between the tan and the target color. The smaller the number means the darker the user's skin tone(typically if lighter skin tone than objective color). Alternatively, the score may measure how far the skin color's numerical value is away from the color black or white's numerical value. The output can be rated as a percentage complete to where the color target is rated as 100% complete. After the score is created, the score, the time amount, the tanning environment, the picture of the user's tan, and current timestamp of the tan session are recorded in a new log 130.

FIG. 3 shows a user interface 200 of an embodiment for accepting user input regarding a recently taken tan session. The tanning environment list 210 allows a user to indicate the tanning method adopted. In this embodiment, three tanning ways are shown, traditional sun tanning, a tanning bed, and applying sunless tanning products. The time amount input 220 receives the time amount taken in the tan session. The “Take Sample” button 230 is used to activate another user interface for receiving a target color and generating a score, such as a Score window in FIG. 3.

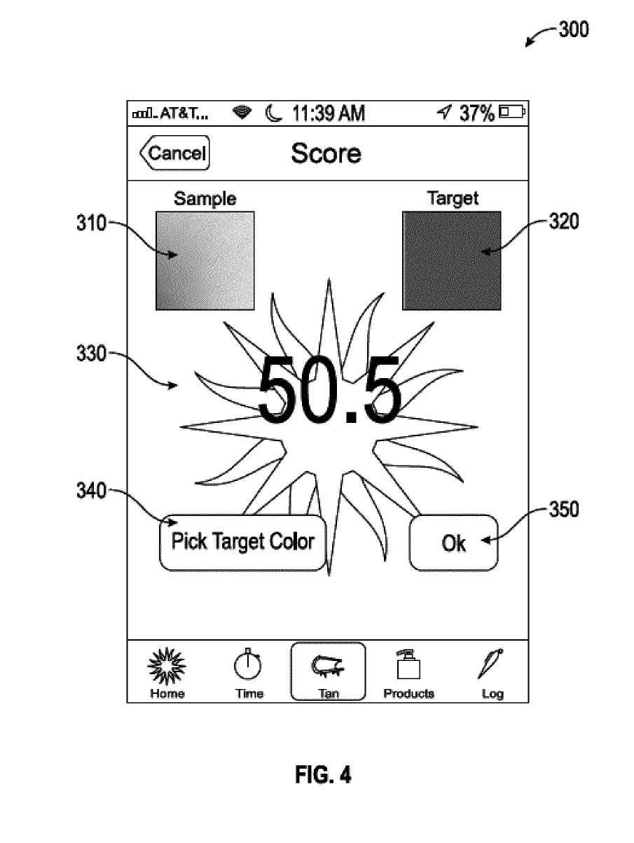

The Score window 300 in FIG. 4 shows a user interface for the user to take a picture 310 of his or her tan and pick a target color 320. The user may select a color that is close to the tan by clicking the “Pick Target Color” button 340, which brings up another user interface such as the Set Color window in FIG. 4. A score 330 is automatically generated based on the target color 320 chosen as percentage or “raw” color scale value. After receiving the required information and a click of the “OK” button 350, the embodiment creates and saves a new tan session.

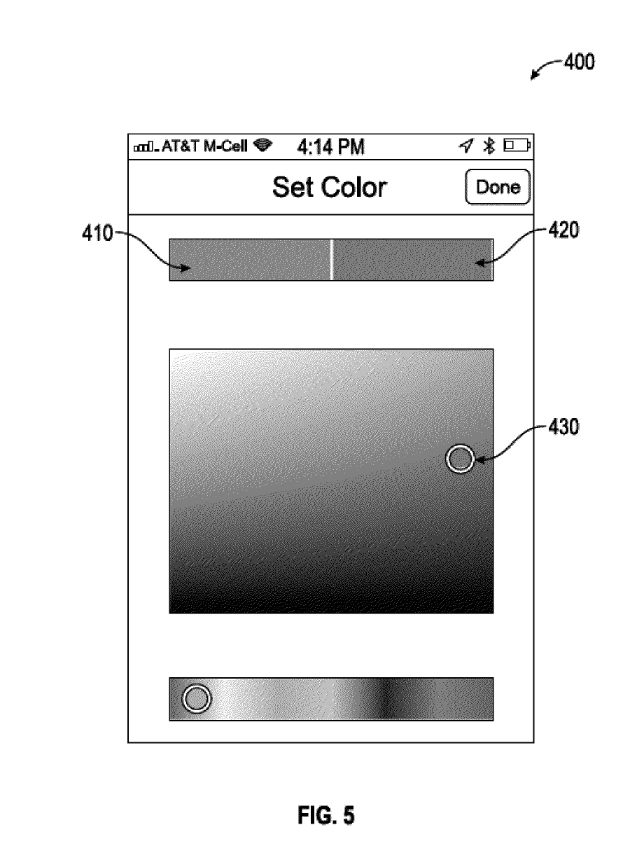

FIG. 5 shows the user interface 400 of an embodiment for choosing a target color. Color block 410 shows the original target color, and color block 420 shows the current color target. The circled area 430 indicates where the user has touched the screen of the mobile device.

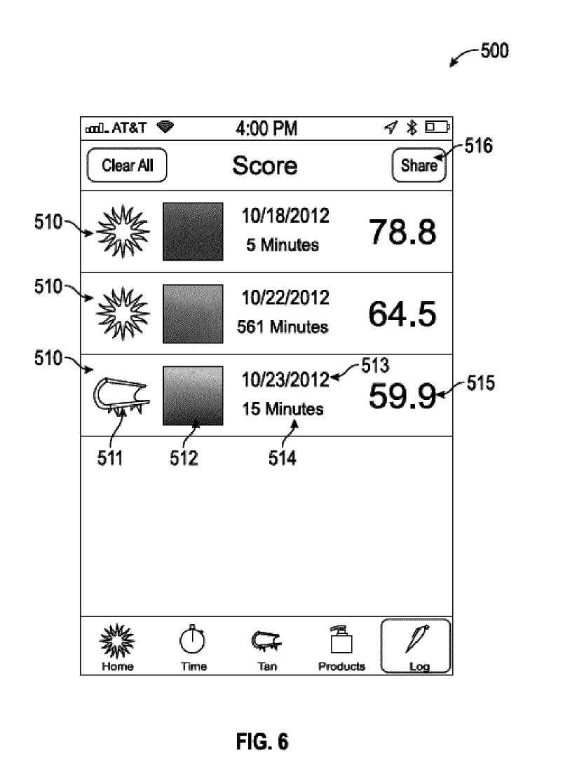

Once a new tan session is saved, it is shown in the Log window 500 of an embodiment in FIG. 6. The tan sessions 510 are displayed in chronological order. Each tan session shows the tanning environment 511, a picture of the user's tan 512, the timestamp 513, the time amount 514, and the score 515. If the user would like to share the latest tan experience, a Share button 516 brings up a list of social media for the user to post the experience.

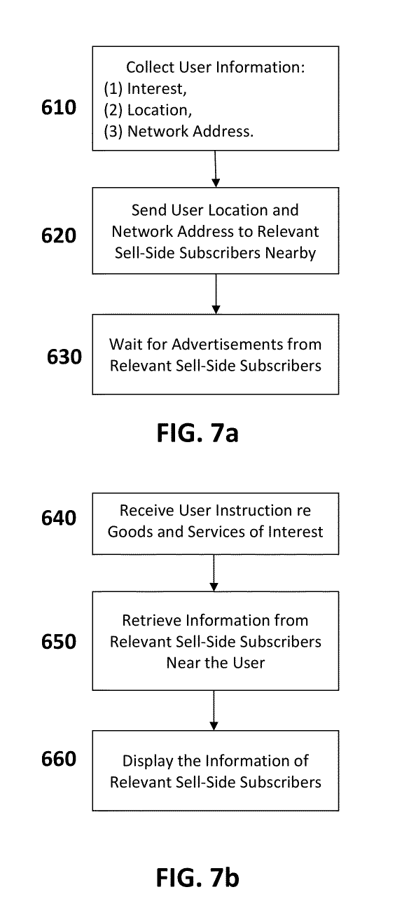

As to bridging provider/consumer gap, the embodiment may adopt methods illustrated in FIGS. 7a & 7b. FIG. 7a shows how an embodiment may allow sell-side subscribers to market their goods and services to a target audience. FIG. 7b illustrates a method for showing users providers of the goods and services of interest.

The system in FIG. 7a contains three modules, a user module of a mobile device, a matching module of a remote server, and a sell-side module of another remote server. In Step 610, the user module prepares the user information, including where the user is in real time and the user's network address for receiving marketing materials. The matching module chooses relevant sell-side subscribers that are located near the user and sends user information to the sell-side modules in Step 620. For the tanning embodiment, the relevant sell-side subscribers are tan salons, tanning product retail stores, and so on.

The matching module may be a remote server as in FIG. 1. The user location information may be used to narrow the geographic area of sell-side subscribers to be matched. User location may be obtained in a variety of ways, but the preferred way is via the global positioning system (GPS). The preferred embodiment has a default value of distance, such as 10 miles, and may allow the user or system software developer to change it.

In Step 630, the user module waits for sell-side modules of the relevant sell-side subscribers to send marketing materials. Alternatively, the user module of an embodiment may retrieve a list of relevant sell-side subscribers nearby and requests contact information or advertisements from relevant sell-side modules.

In FIG. 7b, an embodiment having a user module, a matching module, and a sell-side module helps users to find the goods and services of interest. In Step 640, a user may choose goods and services of interest within a specific geographic area. When receiving a user instruction from the user module, the matching module in Step 650 searches sell-side subscribers within the region that provides the goods and services and shows the result in Step 660.

The matching module may be a third-party searching engine, such as Google. The search result may be a list comprising names, addresses, and phone numbers of matched sell-side subscribers. The result may also be a map with the user location in the center and icons on the map representing matched sell-side subscribers. A sell-side subscriber's information is displayed when its icon is chosen.

While the invention has been described by means of specific embodiments, numerous modifications and variations could be made thereto by those ordinarily skilled in the system development field without departing from the score and spirit of the invention set for the in the claims.

What is claimed is:

1. A device adapted to be interconnected with a user interface for tracking time-lapsed health related issues, the device comprising:
a system host adapted to be interconnected with a user interface, the user interface configured to receive a measured characteristic;
the system host comprising a memory storage module operative to store a base characteristic and measured characteristic;
the system host further comprising a scoring module, the scoring module comprising an executable algorithm for generating a numerical score that reflects the relative distance between the measured characteristic and the base characteristic.

2. The device of claim 1, the measured characteristic selected from the group consisting of skin color, hair color, and teeth color.

3. The device of claim 1, the user interface adapted to be interconnected with a camera and configured to receive digital images from the camera and transmitted to the system host.

4. The device of claim 3, the executable algorithm comprising a comparison of colors.

5. A matching device for notifying a sell-side subscriber of a target consumer, the device comprising:
a matching system host adapted to be interconnected with a subscriber database;
the matching system host comprising a matching module configured to pair a target consumer tracking a time-lapsed heath issue and a sell-side subscriber selling goods or services related to the health issue by matching a location of the target consumer with the sell-side subscriber within a pre-defined radius of the target consumer; and
the matching system host comprising a subscriber database and configured to send contact information of the paired target consumer to the paired sell-side subscriber.

6. The matching device of claim 5, further comprising:
a subscriber system host, the subscriber system host comprising a sell-side module and a client database;
wherein the sell-side module receives the contact information of the paired target consumer and saves the contact information to the client database.

7. The matching device of claim 6, wherein the sell-side module sends a marketing material to the user module of the paired target consumer.

8. A method of tracking time-lapsed health related issues comprising the steps of:
receiving a color;
generating a score that reflects the relative distance between the captured photograph and a base color, target, or goal by comparing the color to the base compare;
generating a timestamp; and
creating a log by saving the score, grade, picture, numerically interpreted value and the timestamp to a database.

9. The method of claim 8, further comprising the steps of:
taking a photograph of a user,
retrieving the color from the photograph, and
saving the picture to the log.

10. A method of notifying a sell-side subscriber of a target consumer comprising the steps of:
receiving contact information from a user interested in tracking a time-lapsed heath issue;
searching a database of sell-side subscribers that sell goods or services related to the health issue;
matching the user with at least one sell-side subscriber; and
providing the contact information to the sell-side subscriber.

11. The method of claim 10, further comprising the steps of:
retrieving a geographic location of the user;
matching the location of the user with at least one sell-side subscriber within a pre-defined or selectable radius of the user; and
providing the contact information access to the sell-side subscriber.
